(19) United States
(12) Patent Application Publication
(10) Pub. No.: US 2009/0297588 A1
(43) Pub. Date: Dec. 3, 2009

(54) ANTIBIOTIC DRESSING FOR THE TREATMENT OF INFECTED WOUNDS

(75) Inventors: MICHAEL RHEINNECKER, Aachen (DE); Rolf Zimmat, Dusseldorf (DE); Lars Steinstraesser, Bochum (DE)

Correspondence Address: INTELLECTUAL PROPERTY / TECHNOLOGY LAW, PO BOX 14329, RESEARCH TRIANGLE PARK, NC, 27709, US

(73) Assignee: Spin'tec Engineering GmbH, Aachen (DE)

(21) Appl. No.: 12/128,466

(22) Filed: May 28, 2008

(51) Int. Cl. A61L 15/16; A61K 38/00; A61P 19/00

(52) U.S. Cl. 424/446; 514/2

(57) ABSTRACT

A silk protein membrane is described which is loaded with an antimicrobial compound and has a substantially non-granular ultrastructure which is (i) substantially devoid of micellar silk fibroin substructures and (ii) substantially devoid of pores when analysed by scanning electron microscopy at 0.2 μm resolution. The antimicrobial compound comprises, in one aspect of the invention, a host defense peptide. The silk protein membrane of the invention can be used in a method for the treatment of wounds and allows the wound dressing to be kept in place after removal of that wound dressing from a wound the wound has less than 10⁵ colony forming units per gram. A method for manufacturing a wound dressing is also disclosed which comprises transferring a cast precursor material and optionally a host defense peptide, into a solid support and then drying the precursor material on the solid support to form a silk protein membrane for use as the wound dressing.

### FIELD OF THE INVENTION

This invention relates to the use of an antimicrobial wound dressing for effective control of wound infections and to a method for the treatment of wound infections

### BACKGROUND OF THE INVENTION

Understanding and managing microbial contamination and colonisation (bioburden) with appropriate treatments is required for the successful healing of chronic wounds (Attinger et al., Plast. Reconstr. Surg. 2006, 177 (Suppl.) 72S). Normally, a chronic wound is in balance with the natural microflora of the skin and mucosal surfaces. However, when the bioburden exceeds a level of greater than 10⁵ bacteria per gram of tissue which can give rise to colony forming units (cfu's) (Robson M C, Surg Clin North Am 1997, 77: 637-650), the chronic wound is considered infected and requires antibiotic treatment. The treatment of the infected chronic wounds represents an enormous burden to health insurers. For example, about 6 million diabetic patients are estimated worldwide to suffer from infection of their chronic diabetic foot wounds each year. The systemic application of antibiotics is considered standard therapy for severe and spreading infections (see Guidelines of the Infectious Disease Society of America, Lipsky et al. 2004). However, for non-spreading chronic foot infections, use of topical antimicrobials instead of the systemic antimicrobials has recently been recommended as best practice (Steed et al., Wound Repair and Regeneration, 2006, 14: 680-692). Unfortunately, the choice of alternatives to the systemic antibiotics is limited. On offer are traditional topical agents such as mafenide acetate (Sulfamylon®) or neomycin which have been found to inhibit the wound-healing process or are relatively toxic to humans and cause contact allergies in patients (Attinger et al., Plast. Reconstr. Surg. 2006, 177 (Suppl.) 72S). In addition, wound dressings loaded with silver ions have failed to demonstrate clinical efficacy (Vermeulen et al., Cochrane Database of Systematic Reviews 2007).

Nature has developed its own class of antibiotics which are present in every life form: antimicrobial cationic host defense peptides (for review read Hancock and Sahl, Nature Biotechnology 2006, 24: 1551-1557). Prominent examples of the host defense peptides (hereinafter defined as HDPs) are alpha-defensins (such as human HNP3), beta-defensins (such as human hBD3), fungal defensins (such as Plectasin), porcine beta-hairpin defensins (such as Protegrin-1), human cathelicidins (such as LL-37), bovine extended defensins (such as Indolicidin) and bacterial cationic peptide antibiotics (such as Nisin, Mersacidin and Polymyxin B and E (colistin)). For a review on colistin read Falagas and Kasiakou, Reviews of Anti-Infective Agents 2005, 40: 1333-1341. Polymyxin B and colistin had been used widely until reports of nephrotoxicity coincided with availability of novel antibiotics in the 1970s. The novel antibiotics then quickly replaced the polymyxins. Since then, there has been a renewed interest in HDPs due to an increasing concern with emergence of multi-drug resistant bacteria (Li et al., Lancet Infect Dis 2006, 6: 589-601). However, despite their excellent activity and broad spectrum, rapid clearance of the HDPs and unfavourable pharmacokinetics, due to proteolytic degradation, has severely restricted their applicability as drugs. Therefore, clinical development of HDPs has focused on topical rather than systemic applications. To increase peptide half-life and improve the resistance to proteolysis, many strategies have been devised. The strategies include integrating HDPs into gels, using different ways of administration or chemical modifying the peptides (see Giuliani et al., 2007, Central European Journal of Biology 2(1), 1-33). Examples for drug release formulations of HDPs are their inclusion in bone cement (for example WO-2000/001427) for use in orthopaedic surgery and in biodegradable gelatine microspheres (Nishikawa et al., J Cell Mol. Med. 2008, doi:10.1111/j. 1582-4934.2008.00341.x).

With respect to the wound healing therapy, however, current slow release formulations of HDPs concur with the generally recommended at least once daily application schedule for antimicrobial ointments or creams to wound beds (Attinger et al, Plast. Reconstr. Surg. 117 (Suppl.): 72S, 2006). For example, the HDP pexiganan, which is currently being developed as a topical cream for treatment of mildly infected diabetic foot ulcers (Macrochem, Inc.) has to be applied to the infected wound bed twice per day (Lipsky et al., “Topical versus systemic antimicrobial therapy for treating infected diabetic foot ulcers: a randomized, controlled, double-blinded, multi-center trial of pexiganan cream”, presented as a scientific exhibit at the Diabetic Foot Global Conference in Los Angeles, Mar. 13-15, 2008). Compositions containing HDPs tethered to a substrate with a specific orientation are also described in WO-A-2007/095393 (MIT). This publication makes no reference to the use of silk protein membranes as slow release carriers or substrates for HDPs.

An example for a membrane made out of regenerated silk and loaded with an artificial HDP derived from insects has been reported by Saido-Sakanaka et al., Journal of Insect Biotechnology and Sericology, 74, 15-20, 2005. However, the authors only demonstrated bacterial killing in a radial diffusion assay and do not teach or demonstrate a method of treatment of infected wounds. There is no reference in the paper or any data provided by the authors on the required or optimal residence time of their antimicrobial silk protein membrane on an infected wound bed. The wound practitioner could not derive from the teachings of Saido-Sakanaka et al. the method of treatment of infective wounds. Saido-Sakanaka et al merely teaches a wound dressing made from the antimicrobial silk protein membrane which needs to be replaced daily by another fresh antimicrobial wound dressing according to the established standard of treating infected wounds. The replacement continues until the infection has been successfully treated (Attinger et al, Plast. Reconstr. Surg. 117 (Suppl.): 72S, 2006).

A disadvantage when using non-native, regenerated silk fibroin as feedstock for casting silk protein membranes like those reported by Saido-Sakanaka et al. is the presence of a distinct granular or globular morphology (so called ultrastructure) when analysed by SEM (scanning electron microscopy). As reported by Jin and Kaplan in Nature, 2003, Vol 424, 1057-1061), this SEM ultrastructure is caused by the aggregation of individual silk fibroin micelles during the drying process of the cast silk fibroin solution. According to Jin and Kaplan, those silk fibroin micelles can also give rise to larger globular structures with diameters of up to 15 μm (see also Nazarov et al. Biomacromolecules 2004, 5, 718-726). Micellar-like morphology has also been demonstrated at high resolution SEM (2 μm scale) by Jin and Kaplan to occur in methanol treated, natural silk protein isolated from the silk glands of B. mori silkworms (Nature, 2003). The silk fibroin micelles have also been reported by G. Freddi et al. in Int J Biomacromolecules 1999, 24: 251-263 as densely packed, roundish particles with diameters of around 200 nm. Because of the granular ultrastructure of regenerated silk protein membranes, it has not been possible yet to manufacture mechanically strong, regenerated silk protein membrane.

Furthermore the silk protein membranes of the prior art have pore sizes which are greater than 200 nm. In order to use the silk protein membranes for biomedical applications and act as an effective physical barrier against antimicrobial and antiviral contamination, it is necessary to have the pore sizes below 200 nm so that viruses and bacteria cannot pass through the silk protein membrane1.

Ideally, for the effective treatment of infected wounds, HDPs should be released continuously from the wound dressing into the infected wound bed thereby eliminating the need for (1) frequent topical application of the drug and (2) frequent time consuming and thereby costly change of the wound dressing. In addition, the wound dressing should also exhibit high mechanical stability to provide adequate protection of the wound, provide an occlusive environment to keep the wound free of particles and toxic wound contaminants and be impermeable to bacteria. The wound dressing should also accelerate or at least have no detrimental effect on the time required for healing of the wound.

There is therefore the need for a novel topical antimicrobial wound dressing which allows efficient infection control through controlled release of HDPs and supports wound closure and healing.

### SUMMARY OF THE INVENTION

The object of the present invention is to improve the method for the treatment of wounds. This object is achieved in one aspect of the invention by using a silk protein membrane as the wound dressing for the treatment of infected wounds loaded with HDPs. The silk protein membrane can be kept in place on the infected wound bed for at least four days and for up to six days (or even longer) without the need of changing the wound dressing (silk protein membrane). This enables for the first time a single use wound dressing therapy with integrated antimicrobial therapeutic activity which eliminates the currently recommended daily change of the wound dressing. This is achieved by extending the retention time of a wound dressing from (currently) one day for at least four days and generally to up to six days (although longer times are not excluded). It will be noted that another advantageous aspect of the invention is that the silk protein membrane has pore sizes smaller than 200 nm for protection of the wounds against microbial and viral contamination.

In a further aspect, a silk protein membrane is described which enables controlled release of HDPs into wounds for the effective treatment of wound infection caused by bacterial, fungal or viral contamination. The silk protein membrane loaded with an antimicrobial compound and has a substantially non-granular ultrastructure which is: (i) substantially devoid of micellar silk fibroin substructures and (ii) substantially devoid of pores. In this context, the term “substantially devoid of micellar silk substructures” and “substantially devoid of pores” means that when analysed by scanning electron microscopy at 0.2 μm resolution, no micellar silk fibroin substructures and almost no pores are detectable. The silk protein membrane appears as a substantially uniform structure.

A method for manufacturing antimicrobial silk protein membranes is also provided.

In one aspect of the invention, the integration of HDPs in the wound dressing takes place before or during the processing of a silk protein solution from a liquid state to a solid state, e.g. during the manufacturing of the silk protein solution into a fiber as described in U.S. Pat. No. 6,858,168 B1 or into the silk protein membrane. In a further aspect, the integration of HDPs in the wound dressing takes place through impregnation or coating after the silk protein solution has been converted from the liquid state to the solid state.

The method of manufacturing the antimicrobial silk protein membranes comprises in one aspect of the invention: casting a silk protein membrane out of a silk protein solution which includes HDPs and then drying the cast silk protein membrane. In another aspect of the invention the silk protein membrane is initially cast out of the silk protein solution (not containing HDPs) and then subsequently loading the silk protein membrane after drying with HDPs.

It will be noted that any natural or artificial silk protein source or feedstock may be used for manufacturing of the antimicrobial silk protein membrane.

### DESCRIPTION OF FIGURES

FIG. 1 shows the method of production of a silk protein membrane for slow release of cationic peptide antibiotics.

FIG. 2 shows a radial diffusion assay with a silk protein membrane, with and without the cationic peptide antibiotic colistin.

FIG. 3 shows the residual content of colistin in a silk protein membrane, during incubation for up to 23 days.

FIG. 4 shows the bacterial counts (cfu/ml) from a microbroth dilution assay against Pseudomonas aeruginosa performed with a colistin silk protein membrane in PBS.

FIG. 5 shows the bacterial counts (cfu/ml) from microbroth dilution assay against Pseudomonas aeruginosa performed with a colistin silk protein membrane in porcine wound fluid.

FIG. 6 shows the bacterial counts (cfu/g) in biopsy porcine tissue obtained from porcine wound infection model with a colistin silk protein membrane.

FIG. 7 shows an apparatus for manufacturing a wound dressing.

FIG. 8 shows a wound dressing according to the invention.

FIG. 9 shows a cross sectional SEM analysis of a silk protein membrane.

### DETAILED DESCRIPTION OF THE INVENTION

For a complete understanding of the present invention and the advantages thereof, reference is now made to the following detailed description taken in conjunction with the Figures.

It should be appreciated that the various aspects of the invention discussed herein are merely illustrative of the specific ways to make and use the invention and do not therefore limit the scope of invention when taken into consideration with the claims and the following detailed description.

The teachings of the cited documents should be incorporated by reference into the description.

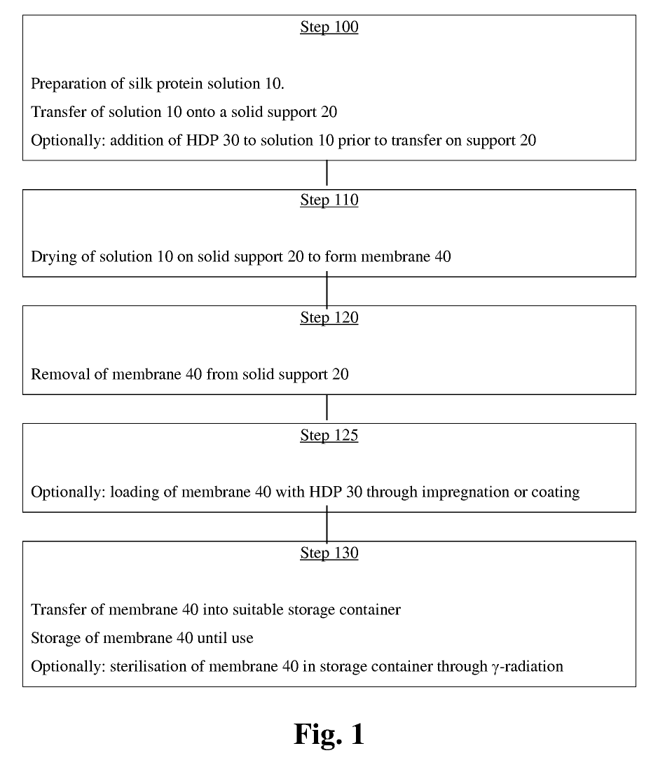

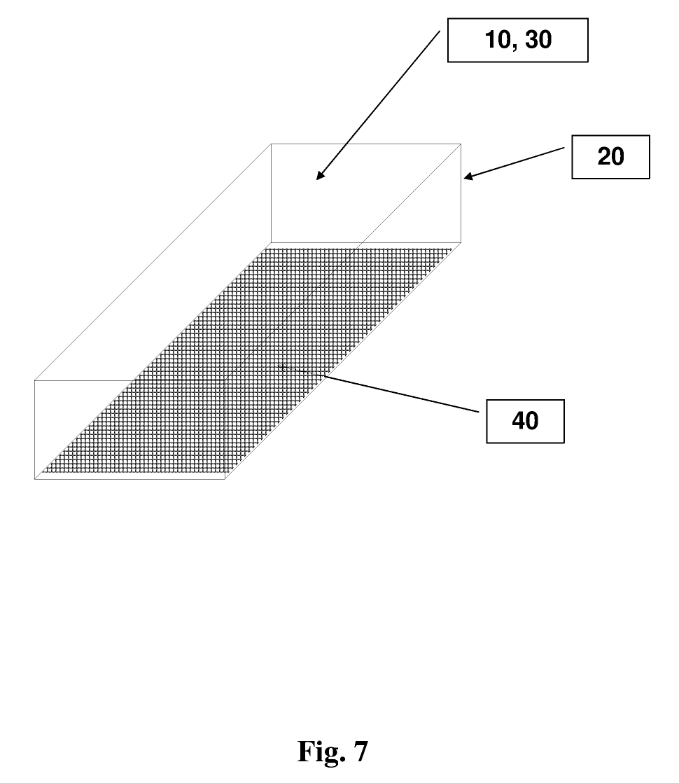

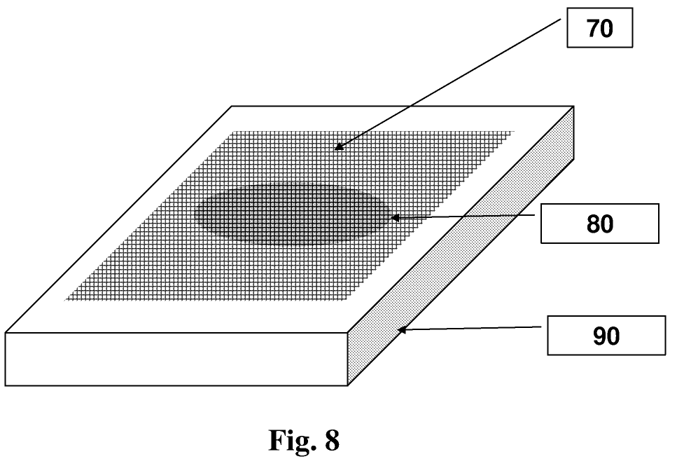

A first method of production of a device for slow release of HDPs is shown in overview in FIG. 1. An apparatus for production of a device for slow release of HDPs is shown in overview in FIG. 7. A device for slow release of HDPs is shown in overview in FIG. 8.

In a first step 100, a silk protein solution 10 is prepared with a silk protein content between 0.3 and 30% (w/w) and a solvent, for example as described in U.S. Pat. No. 7,041,797 B2 and transferred onto a solid support 20. The solid support 20 can be made out of glass or polytetrafluoroethylene (PTFE) or other materials suitable for use with proteins. In one aspect of the invention, HDP 30 may be added to the silk protein solution 10 prior to transfer to the solid support 20.

In the next step 110, the silk protein solution 10 is dried on the solid support 20 to form a silk protein membrane 40. The length of time of drying depends on the protein content of the silk protein solution 10 and the rate of evaporation of the solvent. For drying at room temperature and normal pressure, the drying time of the silk protein solution 10 can vary between 8 h and 48 h for the silk protein solutions with 1-10% silk protein content. The evaporation speed may be varied for example through the use of vacuum techniques or mechanical air blowers.

In the next step 120, the formed silk protein membrane 40 is removed from solid support 20.

In a further aspect of the invention in step 125, the silk protein membrane 40 may be loaded with the HDP 30 through impregnation or surface coating subsequent to the removal of the silk protein membrane 40 from the solid support 20. The exact conditions for the impregnation or surface coating of the silk protein membrane 40 depend on the type of the HDP 30 used. One example was the loading of the silk protein membrane 40 which was 100 μm thick with colistin. It was found that incubation of the silk protein membrane in a colistin solution (100 mg/ml) over night at room temperature was sufficient for integration of colistin throughout the silk protein membrane 40.

In the next step 130, the silk protein membrane 40 is transferred into a suitable container and stored until further use as a wound dressing 70 on a wound 80 on skin 90. Optionally, the silk protein membrane 40 may be sterilised inside the storage container through γ-radiation.

The following examples of specific embodiments for carrying out the present invention are offered for illustrative purposes only and are not intended to limit the scope of the present invention.

### EXAMPLES

### Example 1

### Resistance to Proteolysis of Silk Protein Membranes in Wound Fluid

Silk protein membranes 40 as silk fibroin membranes were made by transferring the protein solution 10 into the solid support 20. The solid support 20 was a casting form made from polytetrafluoroethylene of size 250×110×0.7 mm. The protein solution 10 was produced with the apparatus described in the international application PCT/EP2007/001775. After filling with the protein solution 10, the casting form 20 was left to dry over night at room temperature to yield the silk protein membranes 40 of 80 μm thickness. Without further physical treatment (e.g. heat, mechanical stress) or chemical treatment (e.g. protein denaturing agents, alcohols, cross-linking agents), the silk protein membranes 40 were then cut into rectangular samples (of size 10×3 mm) and transferred individually into 1.5 ml sample tubes. 400 μl of freshly harvested undiluted wound exudates from pig and human wounds were added to each ones of the sample tubes and incubated at 37° C. for 56 hours. The silk protein membranes 40 remained stable and showed no sign of proteolytic degradation by proteases present in the freshly collected wound fluids.

### Example 2

### Ultrastructural Analysis of Silk Protein Membranes by SEM

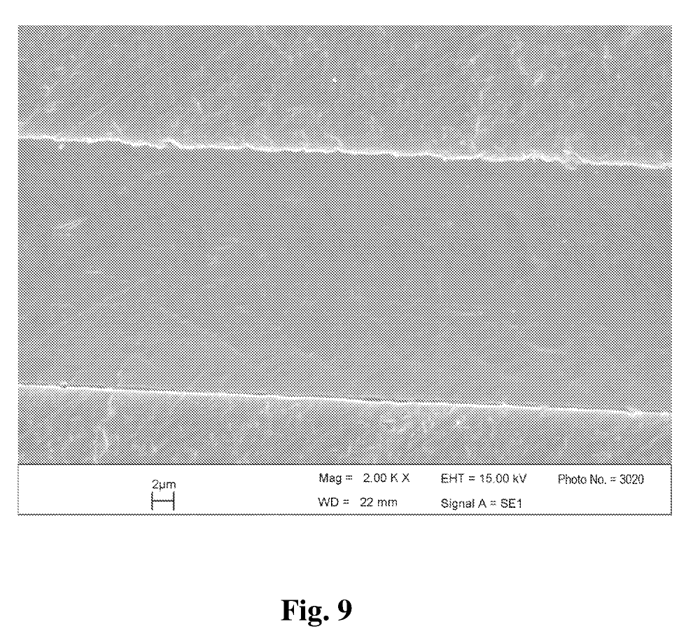

Cross-sections of the silk protein membranes, which were prepared according to the method described in Example 1, were analysed by SEM at high resolution. FIG. 9 (scale bar 2 μm) demonstrates a homogenous SEM ultrastructure of the silk fibroin membrane without detectable pores and without a detectable granular or micellar-like morphology.

### Example 3

### Controlled Release of Colistin Out of Silk Protein Membranes

Round membrane samples 50 with a diameter of 6 mm were stamped out of the silk protein membranes 40 which had been prepared according to Example 1. The round membrane samples 50 were loaded with colistin sulphate (supplied by Carl Roth) through incubation in a colistin solution (10 mg/ml) for 18 hours at room temperature. The incubated round samples 50 were then kept individually in 2 ml solution at room temperature for up to 23 days. Each solution was refreshed every 24 hours in order to simulate wash-out. At defined time points (8 hours and 1, 2, 5, 8, 11, 15, 17, 20, 23 days), the round membrane samples 50 were retrieved, dried and analysed through radial diffusion assay.

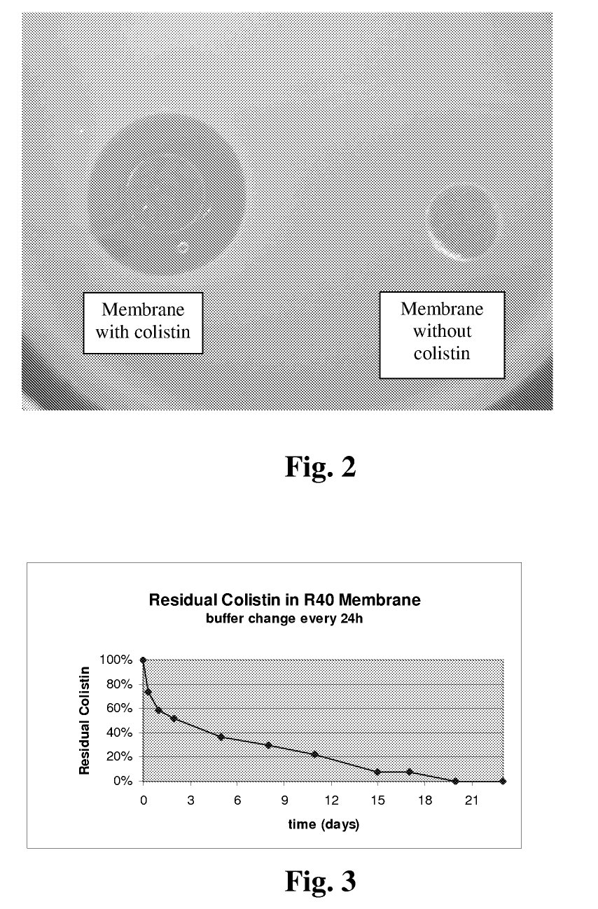

This radial diffusion assay was performed by transferring each one of the round membrane samples 50 onto top-agar plates made by dissolving 32 g LB-Agar Lennox (from Carl Roth) in 400 ml water and adding log-phase E. coli BL21-T1 cells (from Sigma Aldrich) to the agar solution when the temperature of the agar solution cooled down to less than 40° C. As shown in FIG. 2, the antibiotic activity of colistin diffusing out of the round silk protein membranes 50 causes a clear zone (halo) 60 in the bacterial agar which was found to be proportional to the amount of colistin released into the agar. The negative control (i.e. round membrane sample without drug 55) has no antimicrobial activity as indicated through the lack of any clear zone. The residual colistin remaining in each round membrane sample 50 was expressed as a % value of the clear zone 60 of the starting sample (t=0 hours). The half-life for colistin release out of the round membrane samples 50 was approximately 2 days (FIG. 3).

### Example 4

### Confirmation of Antimicrobial Concentration in Microbroth Dilution Assay

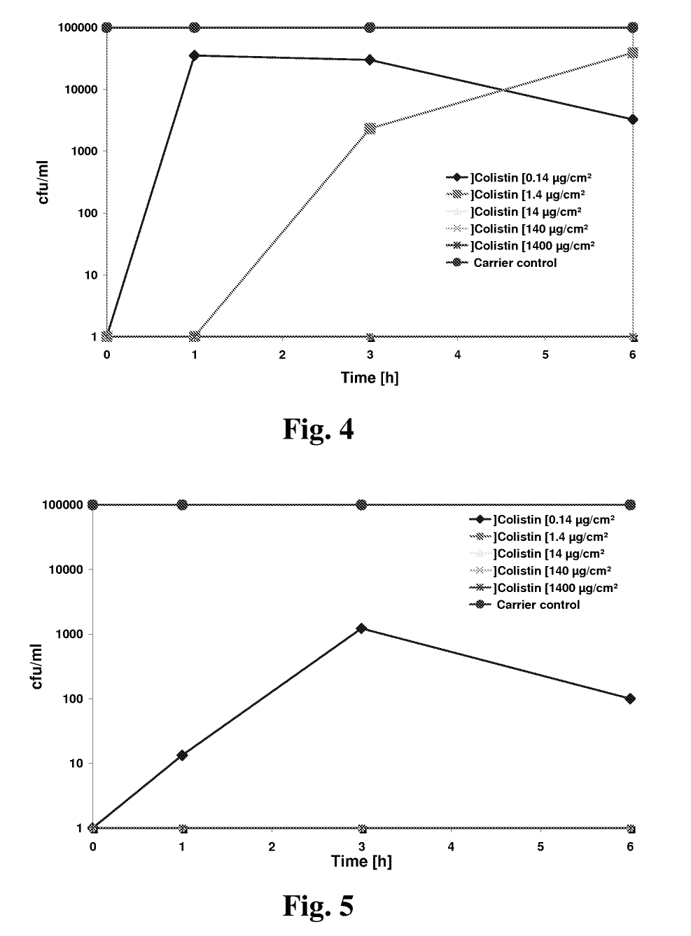

The round membrane samples 50 were produced as described in Example 2 and impregnated with log-scale diluted colistin solutions to yield impregnated round membrane samples containing about 1400, 140, 14, 1.4 and 0.14 μg colistin/cm². To verify that these impregnated round membrane samples release the cationic peptide drug at antimicrobial concentration, the impregnated round membrane samples were tested using a microbroth dilution assay against Pseudomonas aeruginosa in PBS buffer and porcine wound fluid (pWF). The in vitro study demonstrated a concentration dependent antimicrobial effect against P. aeruginosa with complete germ elimination in PBS with round membrane samples 50 impregnated with 1400, 140 and 14 μg/cm² colistin (FIG. 4) and in pWF with round membrane samples 50 impregnated with 1400, 140, 14 and 1.4 μg/cm² colistin (FIG. 5). All of the round membrane samples impregnated with colistin demonstrated lower colony forming unit concentration compared to the corresponding PBS or carrier control.

### Example 5

### Treatment of Wound Infection Through Sustained Release of Colistin in Pig Model

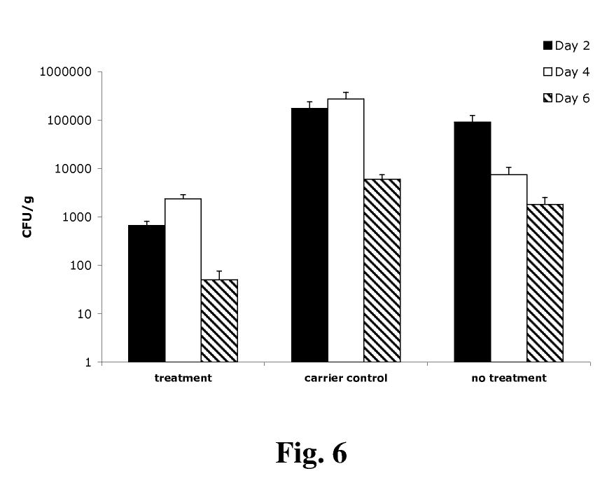

Round membrane samples 50 having 100 Mm thickness and 22 mm diameter were prepared and impregnated to contain about 1.4 mg/cm² colistin as described above. For demonstration of antimicrobial activity of these impregnated round membrane samples 50 in a porcine wound infection model, 12 titanium wound chambers (BO-chamber) were implanted into both flanks of one mini-pig and infected with 5×10⁸ P. aeruginosa. After infection for two days, the wounds were randomized and treated with impregnated round membrane samples 50 containing 1.4 mg/cm² colistin (six impregnated round membrane samples 50 were used for treatment), no colistin (three unimpregnated round membrane samples 50 were used for carrier control) or no treatment (three impregnated round membrane samples 50) as controls. The round membrane samples 50 were left on the wound for 6 days without exchange of the impregnated round membrane samples 50 or cleaning of the wound. Wound fluid and tissue biopsies were collected after 2, 4 and 6 days for quantification of colony forming units (cfu). Within the in vivo trial a cfu reduction of more than 3 log-scales was observed for the treatment group after 2 days (FIG. 6). After 6 days, the treatment group achieved complete bacterial clearance of the infected wound. The carrier control with no colistin integrated in the round membrane samples 50 demonstrated increased bacterial counts due to extensive colonisation of one of the impregnated round membrane samples 50 with bacteria. It is important to note that this animal study was performed with no change of the occlusive dressing throughout the 6 days of treatment and also no additional application of colistin on the occlusive dressing to replenish the colistin reservoir in the impregnated round membrane samples 50.

This in vivo trial demonstrates that the silk protein membranes 40 can act as effective drug depots for controlled release of HDPs into infected wounds. Surprisingly, the topical application of a single one of the silk protein membrane 40, loaded with HDPs 30—in this case colistin sulphate—was sufficient to achieve control of wound infection. The data confirms feasibility of novel, cost-effective treatment modalities for treating infected wounds with the silk protein membranes 40 which are applied as occlusive slow release antibiotic wound dressings and which can be left as occlusive dressings on the wound for several days thereby reducing the number of costly wound dressings and frequency of antibiotic treatments. In addition, the silk protein membranes 40 loaded with HDP 30 required no further addition or reloading of the silk protein membrane 40 with HDPs 30 in order to achieve effective control of the wound infection for a period of several days.

We therefore conclude that silk protein membranes 40 loaded with HDP 30 enable novel treatment modalities for infected wounds, based on effective control of wound infection through controlled release of HDPs, thereby enabling retention of the silk protein membrane of several days on the wound bed.

Reference Numerals

| | |
|---|---|
| 10 | Protein Solution |
| 20 | Solid Support |
| 30 | HDP |
| 40 | Silk Protein Membrane |
| 50 | Round membrane sample |
| 55 | Round membrane sample without drug |
| 60 | Clear Zone |
| 70 | Wound dressing |
| 80 | Wound |
| 90 | Skin |

What is claimed is:

1. A silk protein membrane loaded with an antimicrobial compound and having a substantially non-granular ultrastructure which is:
(i) substantially devoid of micellar silk fibroin substructures and
(ii) substantially devoid of pores
when analysed by scanning electron microscopy at 0.2 μm resolution.

2. The silk protein membrane of claim 1, wherein the antimicrobial compound comprises a host defense peptide.

3. A method for the treatment of wounds comprising:
applying a silk protein membrane loaded with an antimicrobial compound as a wound dressing to the wound, such that after removal of that wound dressing from a wound the wound has less than 10⁵ colony forming units per gram

4. The method of claim 3, wherein the removal of the wound dressing takes place after at the earliest four days.

5. The method of claim 3, wherein the removal of the wound dressing takes place after six days.

6. The method of claim 3, wherein the antimicrobial compound comprises a host defense peptide.

7. The use of a silk protein membrane loaded with an antimicrobial compound as a wound dressing, such that after removal of the wound dressing from a wound the wound has less than 10⁵ colony forming units per gram.

8. A method for manufacturing a wound dressing comprising:
transferring a cast precursor material and optionally a host defense peptide, into a solid support;
drying the precursor material on the solid support to form a membrane; and
removing the membrane to form the wound dressing.

9. The method of claim 8, further comprising loading the membrane with a host defense peptide.

10. The method of claim 9, wherein the loading is through impregnation.

11. The method of claim 9, wherein the loading is through coating.

12. The method of claim 8, wherein the cast precursor material is a silk protein.

13. The method of claim 8, further comprising sterilization by gamma radiation.
